(19) United States
(12) Patent Application Publication
(10) Pub. No.: US 2024/0339188 A1
(43) Pub. Date: Oct. 10, 2024

(54) METHOD FOR CALCULATING A DEGREE OF FATIGUE

(71) Applicant: NEC Corporation, Tokyo (JP)

(72) Inventors: Jingwen LU, Tokyo (JP); Tasuku KITADE, Tokyo (JP); Masanori TSUJIKAWA, Tokyo (JP)

(73) Assignee: NEC Corporation, Tokyo (JP)

(21) Appl. No.: 18/370,215

(22) Filed: Sep. 19, 2023

Related U.S. Application Data
(63) Continuation of application No. 18/030,869, filed on Apr. 7, 2023.

(51) Int. Cl. G16H 20/00; G16H 50/20

(52) U.S. Cl. CPC G16H 20/00; G16H 50/20

(57) ABSTRACT

An information processing device 1X mainly includes an overall fatigue acquisition means 15B, a physical fatigue acquisition means 16B, and a mental fatigue calculation means 17B. The overall fatigue acquisition means 15B is configured to acquire an overall fatigue degree Ft which indicates a degree of overall fatigue of an object person. The physical fatigue acquisition means 16B is configured to acquire a physical fatigue degree Fp which indicates a degree of physical fatigue of the object person. The mental fatigue calculation means 17B is configured to calculate a mental fatigue degree which indicate a degree of mental fatigue of the object person based on the overall fatigue degree Ft and the physical fatigue degree Fp.

This application is a Continuation of U.S. application Ser. No. 18/030,869 filed on Apr. 7, 2023, which is a National Stage Entry of PCT/JP2020/038462 filed on Oct. 12, 2020, the contents of all of which are incorporated herein by reference, in their entirety.

### TECHNICAL FIELD

The present disclosure relates to a technical field of an information processing device, a control method, and a storage medium for calculating a degree of fatigue.

### BACKGROUND ART

There is known a device or system for calculating the degree of fatigue. For example, Patent Literature 1 discloses a technique for measuring the degree of the brain fatigue and the degree of physical fatigue of an object person from information on the electrocardiographic waves detected by electrodes and integrating them by converting them into a common index to thereby determine the degree of overall fatigue.

### CITATION LIST

### Patent Literature

Patent Literature 1: JP 2017-063966A

### SUMMARY

### Problem to be Solved

In general, the physical fatigue degree that is the degree of physical fatigue and the overall fatigue degree that is the degree of overall fatigue can be measured relatively simply and accurately. On the other hand, unfortunately, it is difficult to accurately measure the mental fatigue degree that is the degree of mental fatigue.

In view of the above-described issue, it is therefore an example object of the present disclosure to provide an information processing device, a control method, and a storage medium capable of suitably calculating the degree of mental fatigue.

### Means for Solving the Problem

In one mode of the information processing device, there is provided an information processing device including: an overall fatigue acquisition means configured to acquire an overall fatigue degree which indicates a degree of overall fatigue of an object person; a physical fatigue acquisition means configured to acquire a physical fatigue degree which indicates a degree of physical fatigue of the object person; and a mental fatigue calculation means configured to calculate a mental fatigue degree which indicates a degree of mental fatigue of the object person based on the overall fatigue degree and the physical fatigue degree. The term “overall fatigue degree which indicates a degree of overall fatigue of an object person” herein indicates a degree of fatigue into which physical fatigue and mental fatigue of the object person are integrated.

In one mode of the control method, there is provided a control method executed by a computer, the control method including: acquiring an overall fatigue degree which indicates a degree of overall fatigue of an object person; acquiring a physical fatigue degree which indicates a degree of physical fatigue of the object person; and calculating a mental fatigue degree which indicates a degree of mental fatigue of the object person based on the overall fatigue degree and the physical fatigue degree. The term “computer” herein includes any electronic device (which may be a processor included in the electronic device) and may be configured by multiple electronic devices.

In one mode of the storage medium, there is provided a storage medium storing a program executed by a computer, the program causing the computer to function as: an overall fatigue acquisition means configured to acquire an overall fatigue degree which indicates a degree of overall fatigue of an object person; a physical fatigue acquisition means configured to acquire a physical fatigue degree which indicates a degree of physical fatigue of the object person; and a mental fatigue calculation means configured to calculate a mental fatigue degree which indicates a degree of mental fatigue of the object person based on the overall fatigue degree and the physical fatigue degree.

### Effect

An example advantage according to the present invention is to suitably calculate the degree of mental fatigue.

### BRIEF DESCRIPTION OF THE DRAWINGS

FIG. 1 It illustrates the configuration of a fatigue estimation system in a first example embodiment.

FIG. 2 It illustrates the hardware configuration of am information processing device.

FIG. 3 It is an example of a functional block of the information processing device.

FIG. 4 It is a schematic configuration diagram of a system for generating mental fatigue calculation information.

FIG. 5 It is a first display example of a fatigue confirmation screen image.

FIG. 6 It is a second display example of the fatigue confirmation screen image.

FIG. 7 It is a third display example of the fatigue confirmation screen image.

FIG. 8 It is a fourth display example of the fatigue confirmation screen image.

FIG. 9 It is an example of a flowchart showing the procedure of the fatigue degree calculation process according to the first example embodiment.

FIG. 10 It illustrates the configuration of the fatigue estimation system in a modification.

FIG. 11 It is a functional block diagram of the information processing device according to a second example embodiment.

FIG. 12 It is an example of a flowchart to be executed by the information processing device according to the second example embodiment.

### EXAMPLE EMBODIMENTS

Hereinafter, an example embodiment of an information processing device, a control method, and a storage medium will be described with reference to the drawings.

### First Example Embodiment

### (1) System Configuration

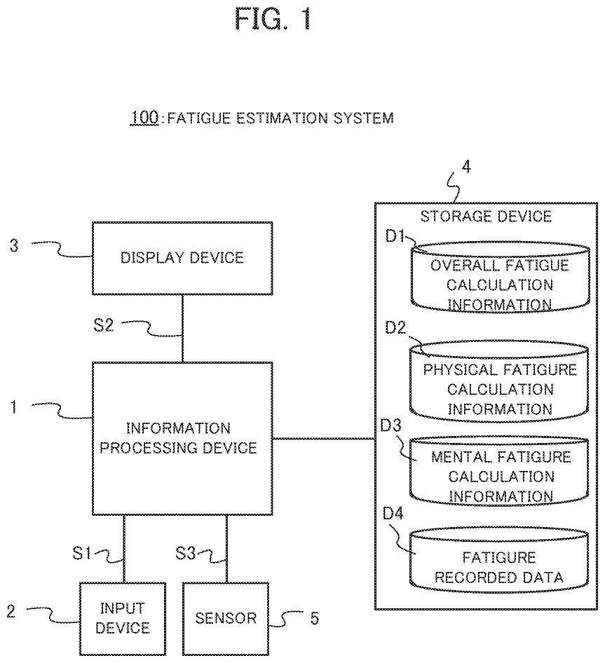

FIG. 1 shows a configuration of a fatigue estimation system 100 according to the first example embodiment. The fatigue estimation system 100 calculates the degree (also referred to as “physical fatigue degree Fp”) of physical fatigue of a person and the degree (also referred to as “overall fatigue degree Ft”) of overall fatigue of the person, and then calculates the degree (also referred to as “mental fatigue degree Fs”) of mental fatigue of the person. The fatigue estimation system 100 mainly includes an information processing device 1, an input device 2, a display device 3, a storage device 4, and a sensor 5.

The information processing device 1 performs data communication with the input device 2, the display device 3, and the sensor 5 through a communication network or through direct wireless or wired communication. The information processing device 1 calculates the physical fatigue degree Fp, the mental fatigue degree Fs, and the overall fatigue degree Ft based on an input signal “S1” supplied from the input device 2, a sensor signal “S3” supplied from the sensor 5, and information stored in the storage device 4. In the present example embodiment, in consideration of the fact that accurate measurement of the mental fatigue degree Fs is generally difficult, the information processing device 1 calculates the physical fatigue degree Fp and the overall fatigue degree Ft of a target person (also referred to as “object person”) of measurement of the degree of fatigue, and then calculates the mental fatigue degree Fs of the object person based on these calculation results. The information processing device 1 generates a display signal “S2” based on the calculation results of the degree of fatigue and supplies the generated display signal S2 to the display device 3.

The input device 2 is one or more interfaces for receiving input from a user, and examples of the input device 2 include a touch panel, a button, a keyboard, a mouse, a voice input device, and the like. The input device 2 supplies the input signal S1 generated based on the input from the user to the information processing device 1. The input signal S1 may be, for example, information indicating a response to a questionnaire regarding fatigue of a target person (also referred to as “object person”) of measurement of the degree of fatigue, or may be input information relating to vital information of the object person or other information required for estimating the fatigue. The input signal S1 may be information indicating results of a body measurement such as a jump for measuring the physical fatigue or the like.

The sensor 5 measures a biological signal or the like of the object person and supplies the measured biological signal or the like to the information processing device 1 as a sensor signal S3. In this instance, the sensor signal S3 may be any biological signal (including vital information) of the object person such as the heart rate, the brain wave, the amount of perspiration, the amount of hormonal secretion, the cerebral blood flow, the blood pressure, the body temperature, the electromyogram, and the respiration rate. The sensor 5 may also be a device configured to analyze the blood of the object person and output the sensor signal S3 indicative of the analysis results. The sensor 5 may be a device configured to perform the body measurement such as jumping for measuring the physical fatigue or the like.

The storage device 4 is one or more memories for storing various information necessary for calculating various kind of the degree of fatigue. The storage device 4 may be an external storage device, such as a hard disk, connected to or embedded in the information processing device 1, or may be a storage medium, such as a flash memory. The storage device 4 may be a server device that performs data communication with the information processing device 1. Further, the storage device 4 may be configured by a plurality of devices.

The storage device 4 stores overall fatigue calculation information D1, physical fatigue calculation information D2, mental fatigue calculation information D3, and fatigue recorded data D4.

The overall fatigue calculation information D1 is information to be required for calculation of the overall fatigue degree Ft, and indicates parameters for calculating the overall fatigue degree Ft based on data (also referred to as “fatigue related data Df”) related to the fatigue of the object person specified from the input signal S1 or the sensor signal S3. The overall fatigue calculation information D1 may be one or more parameters of a general expression for calculating the overall fatigue degree Ft, or may be one or more parameters of a calculation engine obtained by learning a predetermined statistical model or a learning model, similar to the mental fatigue calculation information D3 to be described later. The physical fatigue calculation information D2 is information to be required for calculation of the physical fatigue degree Fp, and indicates parameters or the like for calculating the physical fatigue degree Fp based on the fatigue related data Df. The physical fatigue calculation information D2 may be one or more parameters of a general expression for calculating the physical fatigue degree Fp, or may be one or more parameters of a calculation engine obtained by learning a predetermined statistical model or a learning model, similar to the mental fatigue calculation information D3 to be described later.

The mental fatigue calculation information D3 is information to be required for calculation of the mental fatigue degree Fs, and it indicates one or more parameters of a calculation engine configured to calculate the mental fatigue degree Fs based on the overall fatigue degree Ft and the physical fatigue degree Fp. The model of the calculation engine configured to calculate the mental fatigue Fs may be a statistical model such as a regression model, or may be a model based on machine learning such as a neural network and a support vector machine. In these cases, the mental fatigue calculation information D3 is information indicative of the parameters to be required to configure the calculation engine. For example, when the model of the calculation engine described above is a neural network such as a convolutional neural network, the mental fatigue calculation information D3 includes various parameters such as a layer structure, a neuron structure of each layer, the number of filters and filter sizes in each layer, and a weight for each element of each filter.

The fatigue recorded data D4 is data in which calculation records of the overall fatigue degree Ft, the physical fatigue degree Fp, and the mental fatigue degree Fs are associated with the identification information (object person ID) of the object person, date and time information and the like.

The configuration of the fatigue estimation system 100 shown in FIG. 1 is an example, and various changes may be made to the configuration. For example, the input device 2 and the display device 3 may be integrally configured. In this case, the input device 2 and the display device 3 may be configured as a tablet terminal which includes the information processing device 1 or which is separated from the information processing device 1. Further, the input device 2 and the sensor 5 may be integrally configured. Further, the information processing device 1 may be configured by a plurality of devices. In this case, the plurality of devices constituting the information processing device 1 performs transmission and reception of information necessary for executing preassigned processing among the plurality of devices.

### (2) Hardware Configuration of Information Processing Device

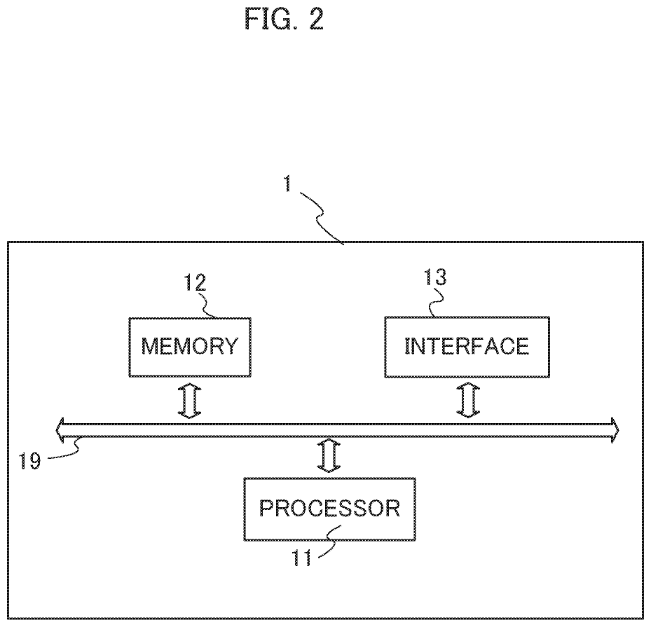

FIG. 2 shows a hardware configuration of the information processing device 1. The information processing device 1 includes a processor 11, a memory 12, and an interface 13 as hardware. The processor 11, memory 12 and interface 13 are connected via a data bus 19.

The processor 11 executes a predetermined process by executing a program stored in the memory 12. The processor 11 is one or more processors such as a CPU (Central Processing Unit), a GPU (Graphics Processing Unit), and a quantum processor.

The memory 12 is configured by various volatile memories and non-volatile memories such as s RAM (Random Access Memory) and s ROM (Read Only Memory). Further, a program executed by the information processing device 1 is stored in the memory 12. The memory 12 is used as a working memory to temporarily store information and the like acquired from the storage device 4. The memory 12 may function as a storage device 4. Similarly, the storage device 4 may function as the memory 12 of the information processing device 1. The program executed by the information processing device 1 may be stored in a storage medium other than the memory 12.

The interface 13 is one or more interfaces for electrically connecting the information processing device 1 to other devices. Examples of the interfaces for connecting the information processing device 1 to other devices include a communication interface, such as a network adapter, for performing wired or wireless transmission and reception of data to and from other devices under the control of the processor 11. In another example, the information processing device 1 may be connected to the other devices by a cable or the like. In this instance, examples of the interface 13 includes a hardware interface which conforms to an USB (Universal Serial Bus), a SATA (Serial AT Attachment), and the like for exchanging data with other devices.

The hardware configuration of the information processing device 1 is not limited to the configuration shown in FIG. 2. For example, the information processing device 1 may include at least one of the input device 2 or the display device 3. Further, the information processing device 1 may be connected to or incorporate a sound output device such as a speaker.

### (3) Functional Block

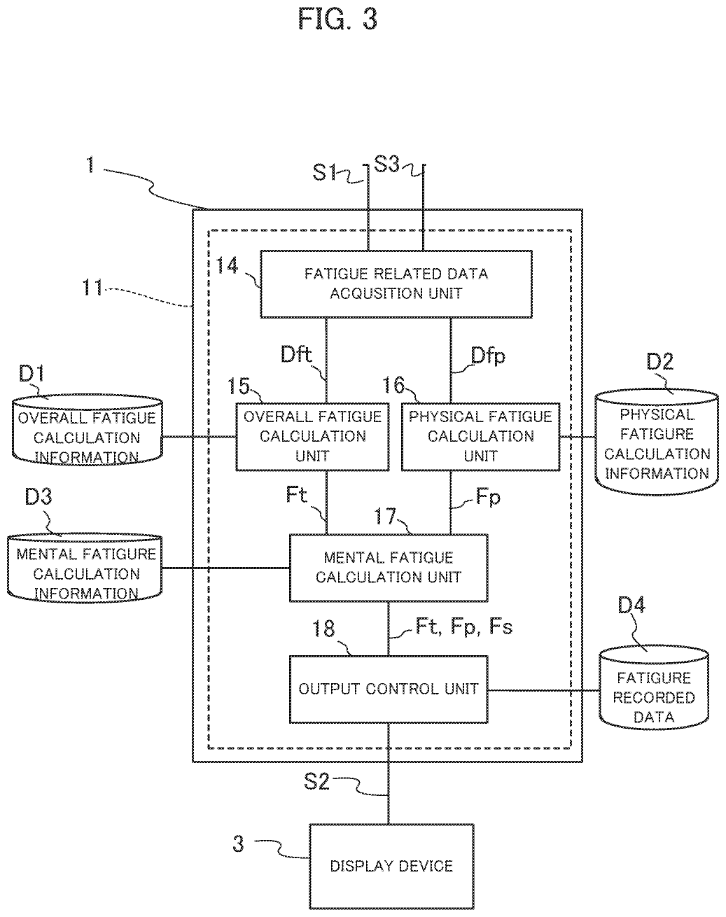

FIG. 3 illustrates an example of the functional block of the information processing device 1 relating to the calculation process of the overall fatigue degree Ft, the physical fatigue degree Fp, and the mental fatigue degree Fs. The processor 11 of the information processing device 1 functionally includes a fatigue related data acquisition unit 14, an overall fatigue calculation unit 15, a physical fatigue calculation unit 16, a mental fatigue calculation unit 17, and an output control unit 18. In FIG. 3, any pairs of blocks which exchange data with each other are connected by solid line, but the pairs of blocks which exchange data with each other is not limited to FIG. 3. The same explanation is also true for the drawings of other functional blocks described below.

The fatigue related data acquisition unit 14 acquires fatigue related data Df to be required for calculating the overall fatigue degree Ft and the physical fatigue degree Fp. Then, the fatigue related data acquisition unit 14 supplies the fatigue related data Df (also referred to as “overall fatigue related data Dft”) to be required for calculating the overall fatigue degree Ft to the overall fatigue calculation unit 15. The fatigue related data acquisition unit 14 supplies the fatigue related data Df (also referred to as “physical fatigue related data Dfp”) to be required for calculating the physical fatigue degree Fp to the physical fatigue calculation unit 16. In this instance, the fatigue related data acquisition unit 14 receives information such as the input signal S1 supplied from the input device 2 and/or the sensor signal S3 supplied from the sensor 5 via the interface 13 and generates the overall fatigue related data Dft and the physical fatigue related data Dfp from the received information. In this instance, the fatigue related data acquisition unit 14 may perform any kind of quantification process on the received information or process of converting the received information into a predetermined index to thereby generate the overall fatigue related data Dft and the physical fatigue related data Dfp. Specific examples of the overall fatigue related data Dft and the physical fatigue related data Dfp will be described later.

The overall fatigue calculation unit 15 refers to the overall fatigue calculation information D1 and calculates the overall fatigue degree Ft from the overall fatigue related data Dft. The overall fatigue calculation unit 15 supplies the calculated overall fatigue degree Ft to the mental fatigue calculation unit 17.

The method of calculating the overall fatigue degree Ft may be a calculation method based on a bioevaluation method, or may be a calculation method based on a subjective evaluation method. For the bioevaluation method, the overall fatigue degree Ft is a biomarker relating to fatigue, and the overall fatigue related data Dft may be data to be required for calculating the biomarker. In another example, the overall fatigue degree Ft is an index obtained by quantifying at least one of HHV-6 (Human Herpes Virus 6) or HHV-7 (Human Herpes Virus 7) reactivated in the object person's saliva, and the overall fatigue related data Dft is data which indicates analysis results of the object person's saliva. There are various methods for biological evaluation of saliva of chronic fatigue patients. For the subjective evaluation method, for example, the overall fatigue degree Ft is a Chalder Fatigue Scale (CFS), and the overall fatigue related data Dft is data indicating the response results of questionnaires to calculate the CFS. Here, the overall fatigue calculation information D1 indicates one or more parameters or the like for converting the overall fatigue related data Ft into the overall fatigue degree Dft based on any one of the above-described calculation methods of the overall fatigue degree Ft. Then, the overall fatigue calculation unit 15 refers to the overall fatigue calculation information D1 and converts the overall fatigue related data Dft into the overall fatigue degree Ft.

The physical fatigue calculation unit 16 refers to the physical fatigue calculation information D2 and calculates the physical fatigue degree Fp from the physical fatigue related data Dfp. Then, the physical fatigue calculation unit 16 supplies the calculated physical fatigue degree Fp to the mental fatigue calculation unit 17.

The calculation method of the physical fatigue degree Fp may be a calculation method based on a bioevaluation method, or may be a calculation method based on a subjective evaluation method, or may be a muscle property-related evaluation method. For the bioevaluation method, for example, the physical fatigue degree Fp is a value of variation in a complete blood count (variation in hemoglobin) for quantitatively evaluating the degree of the fatigue of local muscles, and the physical fatigue related data Dfp is hemoglobin in the blood calculated by near infrared spectroscopy. For the subjective assessment method, the physical fatigue evaluation Fp is an index such as a NRS (Numerical Rating Scale) for evaluating muscle pains, and the physical fatigue related data Dfp is data indicating the response results of the questionnaires for calculating the NRS and the like. For the muscle-related evaluation method, for example, the physical fatigue degree Fp is muscle stiffness, and the physical fatigue related datum Dfp is a measurement value of the biomechanical impedance of the object person's hip. In another instance of the muscle-related evaluation method, the physical fatigue evaluation Fp is a surface myoelectric potential, and the physical fatigue related datum Dfp is a signal of a surface electromyogram measured during exercising time of the object person. Here, the physical fatigue calculation information D2 indicates one or more parameters for converting the physical fatigue related data Fp into the physical fatigue degree Dfp based on any one of the above-described calculation methods of the physical fatigue degree Fp. Then, the physical fatigue calculation unit 16 refers to the physical fatigue calculation information D2 and converts the physical fatigue related data Dfp into the physical fatigue degree Fp.

It is noted that the overall fatigue calculation unit 15 and the physical fatigue calculation unit 16 may further refer to the fatigue related data Df of the object person obtained in the past and stored in the fatigue recorded data D4 or the like to calculate the overall fatigue degree Ft and the physical fatigue degree Fp each of which indicates the degree of relative fatigue to a past state (e.g., a healthy state) of the object person. The overall fatigue degree Ft and the physical fatigue degree Fp may be normalized to be in a predetermined value range (e.g., 0 to 100).

The mental fatigue calculation unit 17 calculates the mental fatigue degree Fs based on the overall fatigue degree Ft calculated by the overall fatigue calculation unit 15 and the physical fatigue degree Fp calculated by the physical fatigue calculation unit 16. In this instance, for example, the mental fatigue calculation unit 17 configures a calculation engine by the mental fatigue calculation information D3 obtained by learning in advance, and acquires the mental fatigue degree Fs outputted by the calculation engine by inputting the overall fatigue degree Ft and the physical fatigue degree Fp into the configured calculation engine. The method of learning the mental-fatigue calculation D3 will be described later. The mental fatigue calculation unit 17 supplies the calculated mental fatigue degree Fs to the outputting control unit 18 together with the overall fatigue degree Ft and the physical fatigue degree Fp.

The output control unit 18 outputs information relating to the overall fatigue degree Ft, the physical fatigue degree Fp, and the mental fatigue degree Fs supplied from the mental fatigue calculation unit 17. For example, the output control unit 18 stores the overall fatigue degree Ft, the physical fatigue degree Fp, and the mental fatigue degree Fs in the storage device 4 as the fatigue recorded data D4 in association with the identification information of the object person and the date and time information. The output control unit 18 generates the display signal S2 for displaying the information on fatigue of the object person on the display device 3 based on the overall fatigue degree Ft, the physical fatigue degree Fp, and the mental fatigue degree Fs and the fatigue recorded data D4 generated in the past. The output control unit 18 supplies the generated display signal S2 to the display device 3 via the interface 13. The information to be displayed on the display device 3 will be described later.

Each component of the overall fatigue calculation unit 15, the physical fatigue calculation unit 16, the mental fatigue calculation unit 17, and the output control unit 18 described in FIG. 3 can be realized, for example, by the processor 11 executing the program stored in the memory 12 or the storage device 4. In addition, the necessary programs may be recorded in any nonvolatile recording medium and installed as necessary to realize each component. Each of these components is not limited to being implemented by software using a program, and may be implemented by any combination of hardware, firmware, and software. Each of these components may also be implemented using user programmable integrated circuitry, such as, for example, FPGA (Field-Programmable Gate Array) or a microcomputer. In this case, the integrated circuit may be used to realize a program functioning as each of the above-described components. Thus, each component may be implemented in hardware other than a processor. The above is the same in other example embodiments to be described later.

### (4) Generation of Mental Fatigue Calculation Information

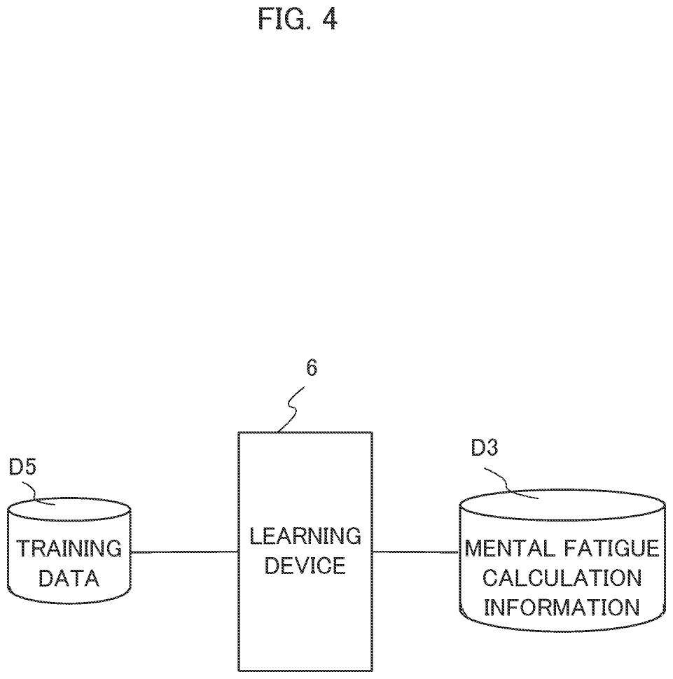

Next, the generation of the mental fatigue calculation information D3 will be described. FIG. 4 is a schematic configuration diagram of a system for generating the mental fatigue calculation information D3. The system includes a learning device 6 which can refer to the training data D5.

For example, the learning device 6 has the same configuration as that of the information processing device 1 shown in FIG. 2, and generates the mental fatigue calculation information D3 based on the training data D5. The learning device 6 may be the information processing device 1, or may be any device other than the information processing device 1.

The training data D5 is, for example, training datasets that are combinations of the overall fatigue degree Ft, the physical fatigue degree Fp, and the mental fatigue degree Fs. The overall fatigue degree Ft, the physical fatigue degree Fp, and the mental fatigue degree Fs in the same combination are measured for the same person at the same timing. The overall fatigue degree Ft and the physical fatigue degree Fp are calculated according to a subjective evaluation method or a bio-evaluation method described in the explanation of the process executed by the overall fatigue calculation unit 15 and the physical fatigue calculation unit 16. The mental fatigue Fs is also similarly calculated according to a subjective evaluation method or a bioevaluation method based on questionnaires or the like. As a bioevaluation method for the mental fatigue degree Fs, for example, there are techniques for identifying the mental fatigue using an EEG (brainwave basic rhythm, P300 waveform).

The learning device 6 performs learning of a calculation engine by using a plurality of combinations of the overall fatigue degree Ft, the physical fatigue degree Fp, and the mental fatigue degree Fs indicated by the training data D5, wherein the calculation engine accepts the overall fatigue degree Ft and the physical fatigue degree Fp as an input and outputs the mental fatigue degree Fs. Here, as an example, it is herein assumed that the above calculation engine is the following multiple regression model with the parameters “w1” and “w2”.

Fs=w1·Ft+w2·Fp

In this instance, the learning device 6 estimates the parameter w1 and the parameter w2 by applying the least squares method or the like using a plurality of combinations of the overall fatigue degree Ft, the physical fatigue degree Fp, and the mental fatigue degree Fs. Then, the learning device 6 generates the estimated parameter w1 and the estimated parameter w2 as the mental fatigue calculation information D3. Thereafter, the mental fatigue calculation unit 17 of the information processing device 1 refers to the mental fatigue calculation information D3 thereby to configure the above-described multiple regression model, and then calculates the mental fatigue degree Fs based on the overall fatigue degree Ft calculated by the overall fatigue calculation unit 15 and the physical fatigue degree Fp calculated by the physical fatigue calculation unit 16.

The model of the calculation engine is not limited to the multiple regression model, and may be any other statistical model or any machine learning model such as a neural network, a support vector machine, and the like. In this case, the learning device 6 uses the overall fatigue degree Ft and the physical fatigue degree Fp extracted from the training data D5 as input data to the calculation engine and uses the mental fatigue degree Fs as the correct answer data, and determines the parameters of the calculation engine such that the error (loss) between the calculation value outputted by the calculation engine when the overall fatigue degree Ft and the physical fatigue degree Fp are inputted thereto and the mental fatigue degree Fs that is the correct answer data are minimized. The algorithm for determining parameters to minimize the loss may be any one of learning algorithms used in machine learning, such as a gradient descent method and an error back propagation method.

### (5) Display Example

Next, a description will be given of display examples of the fatigue confirmation screen image that a screen image to be displayed on the display device 3 under the control of the output control unit 18.

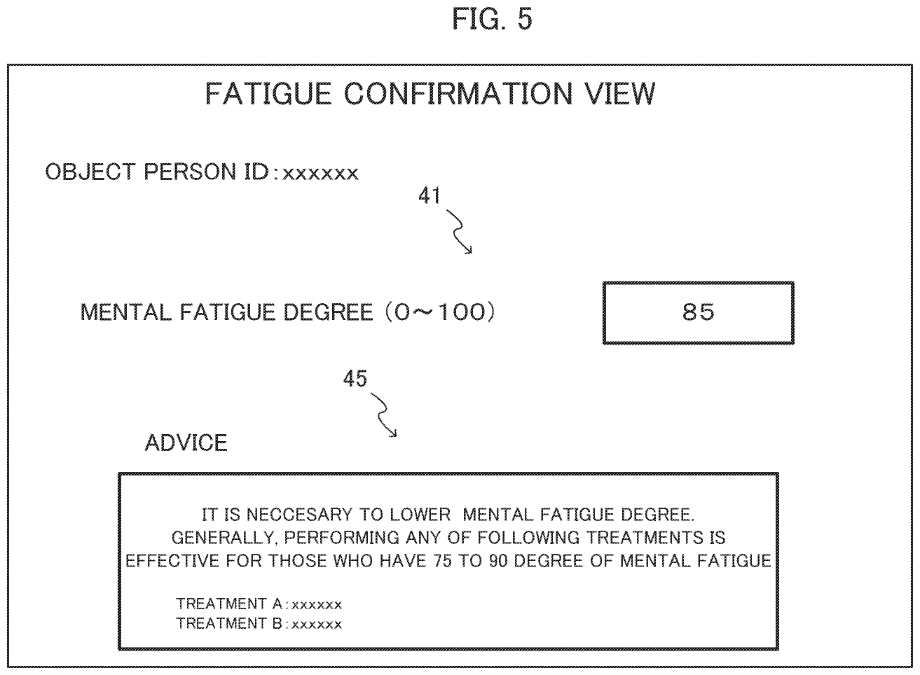

FIG. 5 is a first display example of the fatigue confirmation screen image. The output control unit 18 generates the display signal S2 based on the information supplied from the mental fatigue calculation unit 17 and the information referred to from the storage device 4 and the like, and then supplies the generated display signal S2 to the display device 3 to thereby display the fatigue confirmation screen image on the display device 3. The output control unit 18 at least provides, on the fatigue confirmation screen image according to the first display example, a mental fatigue degree display area 41 and an advice display area 45.

In this instance, the output control unit 18 displays the mental fatigue degree Fs supplied from the mental fatigue calculation unit 17 on the mental fatigue degree display area 41. In this instance, the mental fatigue degree Fs is normalized to range from 0 to 100.

Further, the output control unit 18 displays advice to the object person based on the mental fatigue degree Fs on the advice display area 45. In this case, for example, text information of the advice to be displayed with respect to each delimited value range of the mental fatigue degree Fs is stored in the storage device 4 or the memory 12 in advance, and the output control unit 18 displays the advice on the advice display area 45 by referring to the text information corresponding to the mental fatigue degree Fs. In the example shown in FIG. 5, since the mental fatigue degree Fs belongs to the range of 75 to 90, the output control unit 18 refers to text information to be outputted when the mental fatigue degree Fs belongs to the range of 75 to 90 and displays the advice on the advice display area 45. Here, the output control unit 18 presents some treatments (treatment A and treatment B) for lowering the level of mental fatigue as the advice.

According to the first display example, the information processing device 1 can suitably present the advice according to the mental fatigue Fs and the mental fatigue Fs of the object person to the viewer of the display device 3.

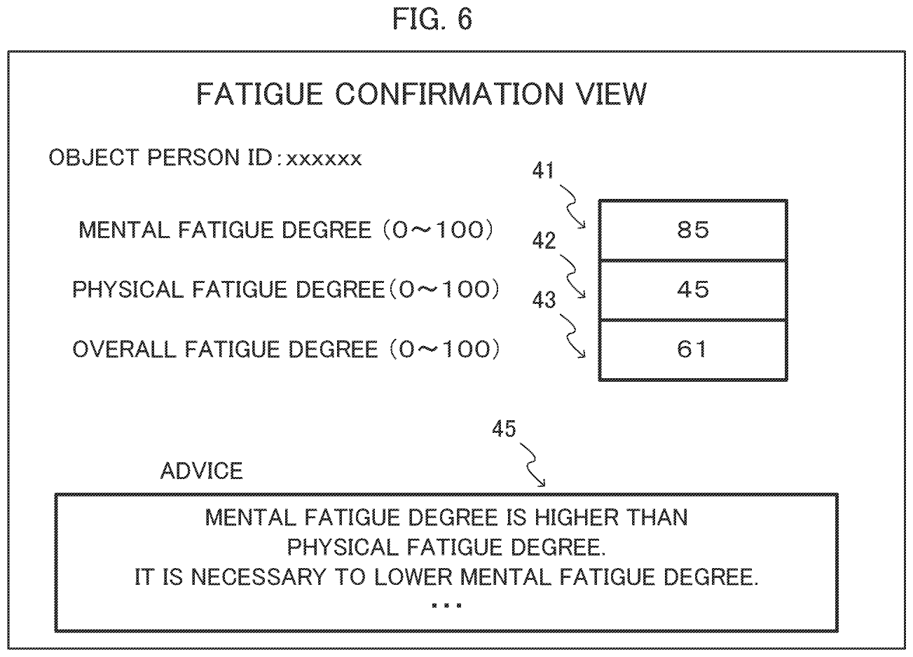

FIG. 6 is a second display example of the fatigue confirmation screen image. The output control unit 18 at least provides, on the fatigue confirmation screen image according to the second display example, a mental fatigue degree display area 41, a physical fatigue degree display area 42, an overall fatigue degree display area 43, and advice display arca 45.

Based on the information supplied from the mental fatigue calculation unit 17, the output control unit 18 displays the mental fatigue degree Fs in the mental fatigue degree display area 41, displays the physical fatigue degree Fp in the physical fatigue degree display area 42, and displays the overall fatigue degree Ft in the overall fatigue degree display area 43. It is noted that the mental fatigue degree Fs, the physical fatigue degree Fp, and the overall fatigue degree Ft are normalized to range from 0 to 100 as an example. Further, the output control unit 18 may display an index (e.g., health level or energy level) which increases with the decrease in the overall fatigue degree Ft, instead of displaying the overall fatigue degree Ft. In this case, for example, the output control unit 18 may display “39” (=“100−Ft”) as the above-mentioned index. Similar indices may be displayed for mental fatigue degree Fs and physical fatigue degree Fp.

Furthermore, the output control unit 18 displays advice on the advice display area 45 based on the comparison result of at least any two of the mental fatigue degree Fs, the physical fatigue degree Fp, and the overall fatigue degree Ft. Here, as an example, the output control unit 18 compares the mental fatigue degree Fs with the physical fatigue degree Fp, and displays the information indicating that the mental fatigue degree Fs needs to be lowered and the treatments for lowering the mental fatigue degree Fs in the advice display area 45. In this case, in the same way as in the first display example, the output control unit 18 may display one or more treatments corresponding to the degree of fatigue that needs to be lowered on the advice display arca 45.

According to the second display example, the output control unit 18 can compareably present the mental fatigue degree Fs, the physical fatigue degree Fp, and the overall fatigue degree Ft of the object person to the viewer of the display device 3 and suitably present the advice as to whether the physical care or the mental care should be prioritized.

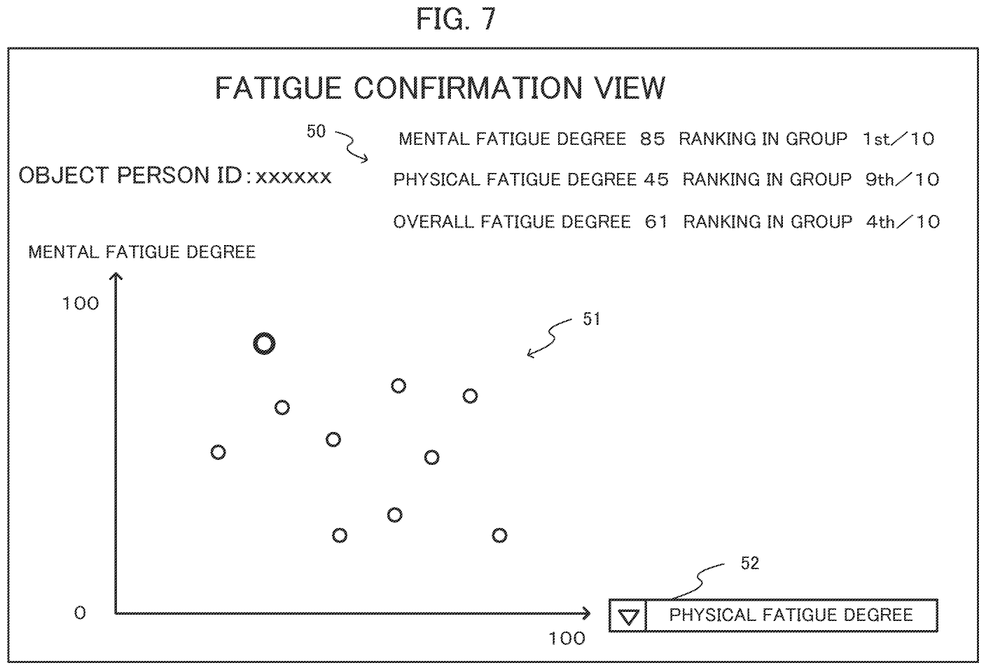

FIG. 7 is a third display example of the fatigue confirmation screen image. The output control unit 18 at least provides, on the fatigue confirmation screen image according to the third display example, a fatigue degree comparison information display area 50, and a fatigue degree distribution display area 51.

In the third display example, the output control unit 18 extracts the information on the fatigue degree of all members other than the object person in the group to which the object person belongs from the fatigue recorded data D4, and displays, on the fatigue confirmation screen image, information on the comparison results between the fatigue degree of the other members and the fatigue degree of the object person. Here, the group may be a group for each workplace, or may be a group according to the classification based on any attribute such as job type, gender, and the like.

Specifically, on the basis of each fatigue degree of the object person supplied from the mental fatigue calculation unit 17 and each fatigue degree of the other members extracted from the fatigue recorded data D4, the output control unit 18 displays each value of the mental fatigue degree Fs, the physical fatigue degree Fp, and the overall fatigue degree Ft and the ranking within the group, on the fatigue degree comparison information display area 50. Further, the output control unit 18 displays a scatter diagram indicative of the distribution of the fatigue degrees of the members in the target group on the fatigue degree distribution display area 51, wherein the scatter diagram has the vertical axis corresponding to the mental fatigue Fs and the horizontal axis corresponding to the physical fatigue Fp. In the above distribution, the output control unit 18 highlights and displays the plot corresponding to the target person by bold circle. The output control unit 18 displays a pull-down menu 52 for specifying an index of the horizontal axis of the distribution. The pull-down menu 52 accepts a switching operation of the horizontal axis between the physical fatigue degree Fp and the overall fatigue degree Ft.

Thus, in the third display example, the output control unit 18 can let the viewer of the display device 3 suitably grasp the relative fatigue degree of the object person in the group.

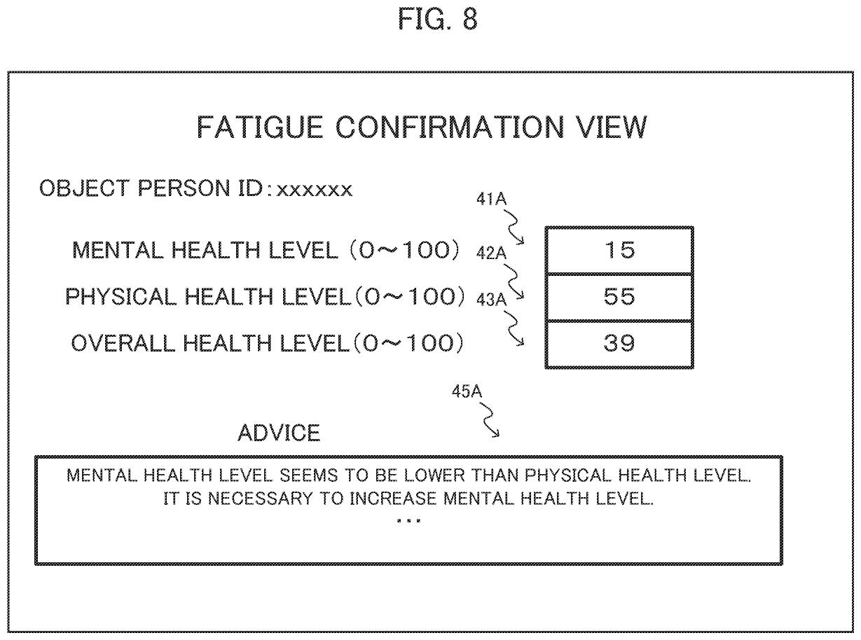

FIG. 8 is a fourth display example of the fatigue confirmation screen image. The output control unit 18 at least provides, on the fatigue confirmation screen image according to the fourth display example, a mental health level display area 41A, a body health level display area 42A, an overall health level display area 43A, and an advice display area 45A.

In the fourth display example, the output control unit 18 displays the mental health level, which is an index based on the mental fatigue degree Fs, on the mental health level display area 41A, displays the physical health level, which is an index based on the physical fatigue degree Fp, on the physical health level display area 42A, and displays the overall health level, which is an index based on the overall fatigue degree Ft, on the overall health level display area 43A. Here, the mental health level is set to increase with the decrease in the mental fatigue degree Fs, the physical health level is set to increase with the decrease in the physical fatigue degree Fp, the overall health level is set to increase with the decrease in the overall fatigue degree Ft. In this way, in the fourth display example, the output control unit 18 can suitably prompt physical or mental care by displaying the information based on the health level (energy level) that is an index having a negative correlation with the fatigue degree.

### (6) Processing Flow

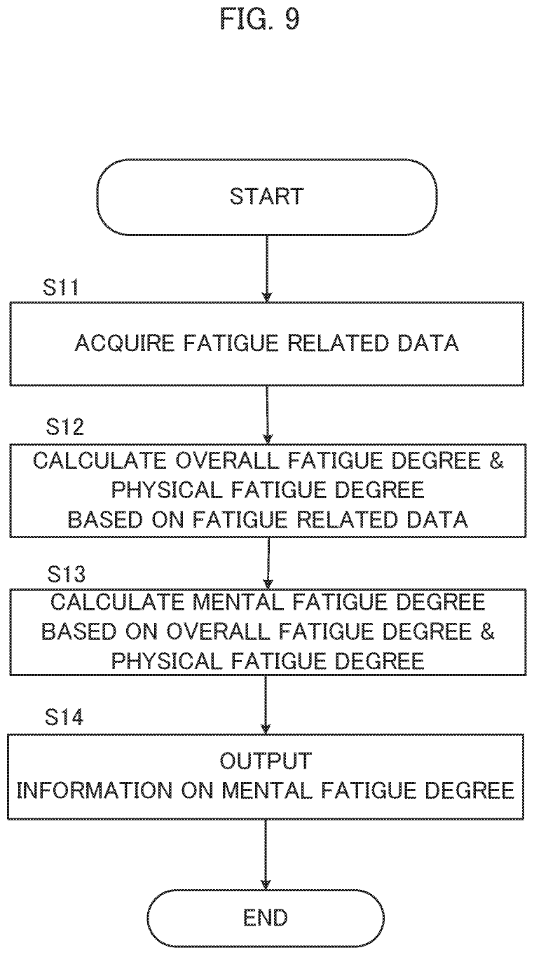

FIG. 9 is an example of a flowchart illustrating a procedure of the fatigue degree calculation process that is executed by the information processing device 1 according to the first example embodiment. The information processing device 1 executes the processing according to the flowchart shown in FIG. 9, for example, when there is at least one input of the input signal S1 or the sensor signal S3 to be required for the fatigue degree calculation process.

First, the information processing device 1 acquires fatigue related data Df (step S11). In this instance, the fatigue related data acquisition unit 14 of the information processing device 1 acquires the overall fatigue related data Dft and the physical fatigue related data Dfp, respectively, based on the input signal S1 supplied from the input device 2 or the sensor signal S3 supplied from the sensor 5, for example.

Next, the information processing device 1 calculates the overall fatigue degree Ft and the physical fatigue degree Fp based on the fatigue related data Df (step S12). In this instance, the overall fatigue calculation unit 15 of the information processing device 1 calculates the overall fatigue degree Ft from the overall fatigue related data Dft by referring to the overall fatigue calculation information D1 while the physical fatigue calculation unit 16 of the information processing device 1 calculates the physical fatigue degree Fp from the physical fatigue related data Dfp by referring to the physical fatigue calculation information D2.

Next, the information processing device 1 calculates the mental fatigue degree Fs based on the overall fatigue degree Ft and the physical fatigue degree Fp (step S13). In this instance, the mental fatigue calculation unit 17 of the information processing device 1 configures the calculation engine by referring to the mental fatigue calculation information D3, and acquires the mental fatigue degree Fs by inputting the overall fatigue degree Ft and the physical fatigue degree Fp to the calculation engine.

Then, the information processing device 1 outputs the information on the mental fatigue degree Fs (step S14). In this instance, for example, the output control section 18 of the information processing device 1 generates a display signal S2 for displaying a fatigue confirmation screen image according to any one of modes as shown in FIGS. 5 to 8 by referring to the calculation results at step S12 and step S13, and the fatigue recorded data D4 stored in the storage device 4.

Then, the output control unit 18 causes the display device 3 to display the fatigue confirmation screen image by transmitting the generated display signal S2 to the display device 3.

### (7) Modifications

Next, a description will be given of each modification suitable for the above example embodiment. The following modifications may be applied to the above-described example embodiment in any combination.

### First Modification

The information processing device 1 may cause a sound output device (not shown) to output the information on the degree of fatigue, instead of causing the display device 3 to display the information.

In this case, for example, in the same situation as shown in FIG. 5, the information processing device 1 supplies a sound signal for outputting “degree of mental fatigue is 85” to the sound output device based on the calculated mental fatigue degree Fs. Further, the information processing device 1 similarly supplies the voice signal of the advice to be displayed on the advice display area 45 to the sound output device, thereby to cause the sound output device to output the voice of the advice. According to this mode, the information processing device 1 can suitably notify the user of information on the degree of fatigue.

### Second Modification

Instead of using the calculation engine, the mental fatigue calculation unit 17 may calculate the mental fatigue degree Fs by referring to table information. In this case, the mental fatigue calculation informational D3 is, for example, a look-up table indicative of the mental fatigue degree Fs corresponding to each possible combination of the physical fatigue degree Fp and the overall fatigue degree Ft. Then, the mental fatigue calculation unit 17 determines the mental fatigue degree Fs on the basis of the overall fatigue degree Ft calculated by the overall fatigue calculation unit 15, the physical fatigue degree Fp calculated by the physical fatigue calculation unit 16, and the above-described look-up table.

### Third Modification

The physical fatigue calculation unit 16 may calculate a plurality of types of the physical fatigue degree Fp. For example, the physical fatigue degree Fp indicates a plurality of index values of the physical fatigue degree calculated by different calculation methods. Similarly, the overall fatigue calculation unit 15 may calculate a plurality of types of the overall fatigue degree Ft. In this case, for example, the overall fatigue degree Ft indicates a plurality of index values of the overall fatigue degree calculated by different calculation methods.

In these cases, the mental fatigue calculation unit 17 calculates the mental fatigue degree Fs from the respective fatigue degrees calculated by the overall fatigue calculation unit 15 and the physical fatigue calculation unit 16. For example, in this case, the calculation engine is learned to calculate the mental fatigue degree Fs when a predetermined number of values of the overall fatigue degree Ft and a predetermined number of values of the physical fatigue degree Fp are inputted thereto. Then, the mental fatigue calculation unit 17 constructs the above-described calculation engine by referring to the mental fatigue calculation information D3 corresponding to the above-described parameters of the calculation engine, and calculates the mental fatigue degree Fs from the respective fatigue degrees calculated by the overall fatigue calculation unit 15 and the physical fatigue calculation unit 16. In this instance, the mental fatigue calculation unit 17 may calculate a single value of the mental fatigue degree Fs or may calculate plural values of the mental fatigue degree Fs. In another example, the mental fatigue calculation unit 17 calculates a representative value, such as an average value, of the values of the overall fatigue degree Ft calculated by the overall fatigue calculation unit 15 and a representative value, such as an average value, of the values of the physical fatigue degree Fp calculated by the physical fatigue calculation unit 16. In the case of calculating the representative value described above, the mental fatigue calculation unit 17 may perform the normalizing process as the pre-processing so that the values to be used for calculating the representative value have the same value range. Then, the mental fatigue calculation unit 17 calculates the mental fatigue degree Fs by referring to the mental fatigue calculation information D3 on the basis of the representative value of the overall fatigue degree Ft and the representative value of the physical fatigue degree Fp. In this case, in the same way as in the above-described example embodiment, the calculation engine is learned to calculate the mental fatigue degree Fs when a single value of the overall fatigue degree Ft and a single value of the physical fatigue degree Fp are inputted thereto.

Thus, even when at least one of the physical fatigue degree Fp or the overall fatigue degree Ft indicates a plurality of index values, the mental fatigue calculation unit 17 can suitably calculate the mental fatigue degree Fs.

### Fourth Modification

The mental fatigue calculation unit 17 may calculate the mental fatigue degree Fs by further considering the attribute(s) of the object person.

In this case, for example, the mental fatigue calculation information D3 for each category classified according to the attribute is stored in the storage device 4. When calculating the mental fatigue degree Fs, the mental fatigue calculation unit 17 acquires the attribute of the object person based on the input signal S1 or the like, and extracts the mental fatigue calculation information D3 corresponding to the category of the acquired attribute from the storage device 4, and calculates the mental fatigue degree Fs from the overall fatigue degree Ft and the physical fatigue degree Fp using a calculation engine configured based on the extracted mental fatigue calculation information D3. The category in this case may be determined based on one or more attributes such as gender, age, occupation, and annual yield. Further, in the learning stage of the mental fatigue calculation information D3, the training data D5 includes the attribute information of the object person, and the learning device 6 performs learning of the calculation engine of the mental fatigue degree Fs and generating of the mental fatigue calculation information D3 for each category based on the combinations of the overall fatigue degree Ft, the physical fatigue degree Fp, and the mental fatigue degree Fs classified based on the attribute information. Each mental fatigue calculation information D3 is then associated with information on the corresponding category of the attribute.

According to this modification, the mental fatigue calculation unit 17 can calculate a more accurate mental fatigue degree Fs in consideration of the attribute of the object person.

### Fifth Modification

The fatigue estimation system 100 may be a server client model.

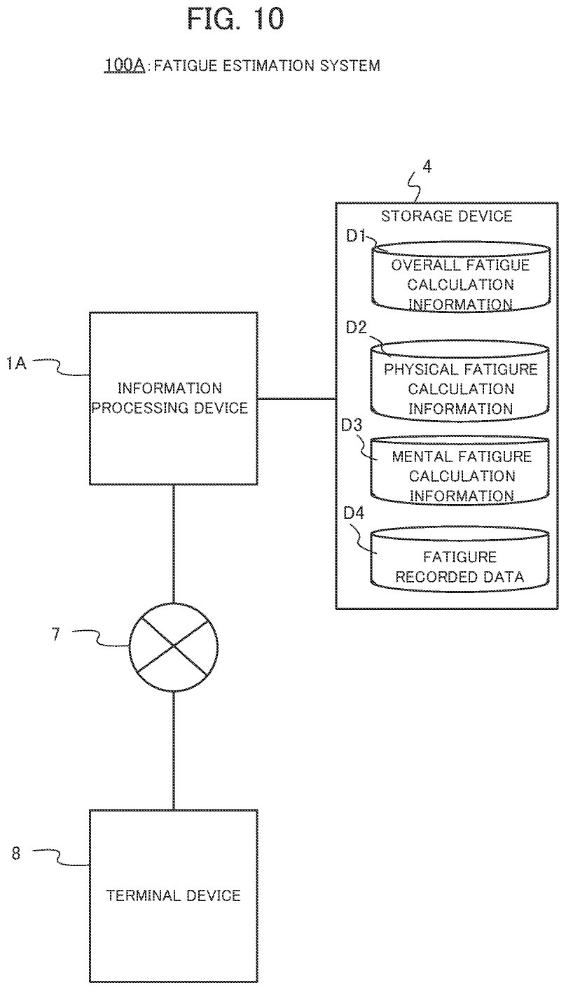

FIG. 10 shows the configuration of a fatigue estimation system 100A according to the fifth modification. As shown in FIG. 10, the fatigue estimation 100A mainly includes an information processing device 1A that functions as a server, a storage device 4 that stores data to be required for the fatigue estimation process, and a terminal device 8 that functions as a client. The information processing device 1A and the terminal device 8 communicates data with each other via the network 7.

The terminal device 8 is a terminal having an input function, a display function, and a communication function, and functions as the input device 2 and the display device 3 shown in FIG. 1. The terminal device 8 may be, for example, a personal computer, a tablet-type terminal, a PDA (Personal Digital Assistant), or the like. The terminal device 8 transmits to the information processing device 1A information based on a user input or a biological signal outputted by one or more sensors (not shown). The terminal device 8 may transmit information corresponding to the fatigue related data Df to the information processing device 1A.

The information processing device 1A has the same configuration as the information processing device 1 shown in FIG. 1. Then, the information processing device 1A receives information, that is to be acquired by the information processing device 1 shown in FIG. 1 from the input device 2 and the sensor 5, from the terminal device 8 via the network 7. Then, the information processing device 1A transmits the display signal S2 indicating the information on the calculated degree of fatigue to the terminal device 8 through the network 7 after the execution of the fatigue degree calculation process. Thus, the information processing device 1A can calculate the respective degrees of fatigue including the mental fatigue degree Fs and suitably cause the terminal device 8 to display the information indicative of the calculated degrees of fatigue.

### Second Example Embodiment

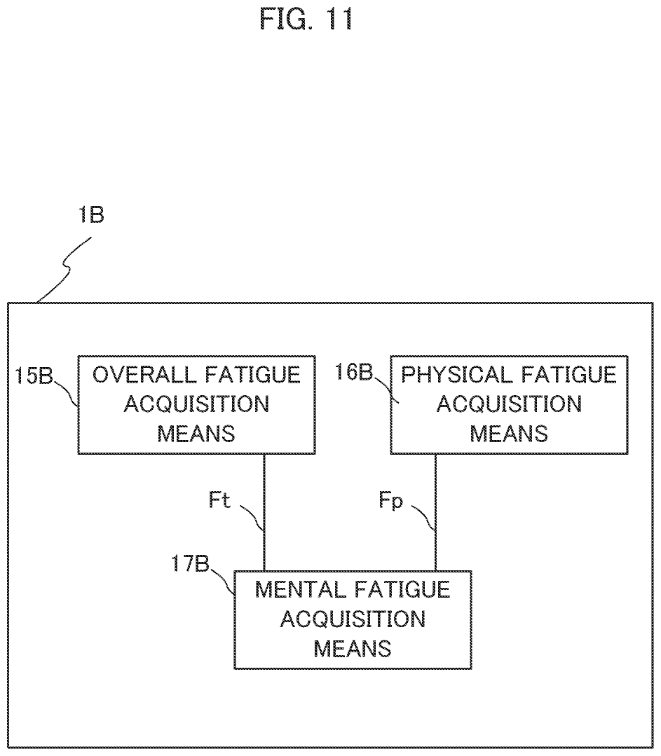

FIG. 11 is a functional block diagram of an information processing device 1B according to a second example embodiment. The information processing device 1B mainly includes an overall fatigue acquisition means 15B, a physical fatigue acquisition means 16B, and a mental fatigue calculation means 17B.

The overall fatigue acquisition means 15B is configured to acquire an overall fatigue degree “Ft” which indicates a degree of overall fatigue of an object person. Examples of the overall fatigue acquisition unit 15B include the overall fatigue calculation unit 15 in the first example embodiment. In another example, the overall fatigue acquisition means 15B is configured to receive the overall fatigue degree Ft calculated by an external device having a function corresponding to the overall fatigue calculation unit 15 in the first example embodiment from the external device. In yet another example, the overall fatigue acquisition means 15B is configured to receive a user input that specifies the overall fatigue degree Ft by an input device and thereby acquire the value specified by the user as the overall fatigue degree Ft.

The physical fatigue acquisition means 16B is configured to acquire a physical fatigue degree “Fp” which indicates a degree of physical fatigue of the object person. Examples of the physical fatigue acquisition means 16B include the physical fatigue calculation unit 16 in the first example embodiment. In another example, the physical fatigue acquisition means 16B is configured to receive the physical fatigue degree Fp calculated by an external device having a function corresponding to the physical fatigue calculation unit 16 in the first example embodiment from the external device. In yet another example embodiment, the physical fatigue acquisition means 16B is configured to receive a user input that specifies the physical fatigue degree Fp by an input device and thereby acquire the value specified by the user as the physical fatigue degree Fp.

The mental fatigue calculation means 17B is configured to calculate a mental fatigue degree which indicates a degree of mental fatigue of the object person based on the overall fatigue degree Ft and the physical fatigue degree Fp. Examples of the mental fatigue calculation means 17B include the mental fatigue calculation unit 17 in the first example embodiment.

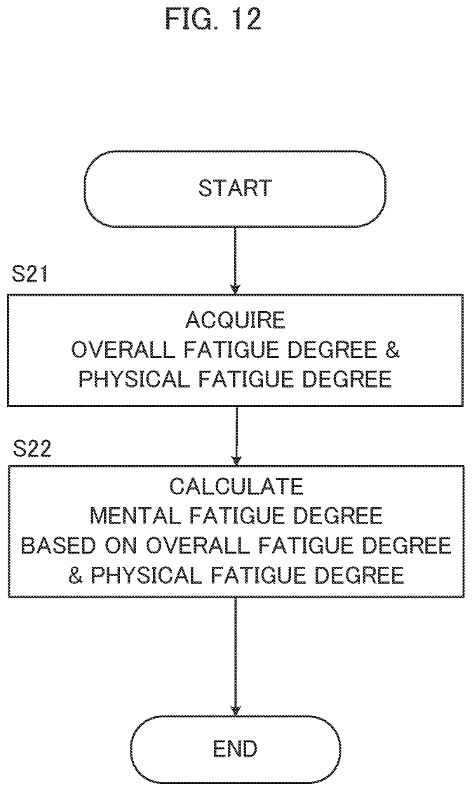

FIG. 12 is an example of a flowchart that is executed by the information processing device 1B in the second example embodiment. First, the overall fatigue acquisition means 15B acquires an overall fatigue degree Ft which indicates a degree of overall fatigue of an object person, and the physical fatigue acquisition means 16B acquires a physical fatigue degree Fp which indicates a degree of physical fatigue of the object person (step S21). Then, the mental fatigue calculation means 17B is configured to calculate a mental fatigue degree which indicates a degree of mental fatigue of the object person based on the overall fatigue degree Ft and the physical fatigue degree Fp (step S22).

The information processing device 1B according to the second example embodiment can suitably calculate the mental fatigue degree of the object person.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### [Supplementary Note 1]

An information processing device comprising:
an overall fatigue acquisition means configured to acquire an overall fatigue degree which indicates a degree of overall fatigue of an object person;
a physical fatigue acquisition means configured to acquire a physical fatigue degree which indicates a degree of physical fatigue of the object person; and
a mental fatigue calculation means configured to calculate a mental fatigue degree which indicates a degree of mental fatigue of the object person based on the overall fatigue degree and the physical fatigue degree.

### [Supplementary Note 2]

The information processing device according to Supplementary Note 1, further comprising
a fatigue related data acquisition means configured to acquire fatigue related data related to fatigue of the object person,
wherein the overall fatigue acquisition means is configured to calculate the overall fatigue degree based on at least a part of the fatigue related data, and
wherein the physical fatigue acquisition means is configured to calculate the physical fatigue degree based on at least a part of the fatigue related data.

### [Supplementary Note 3]

The information processing device according to Supplementary Note 1 or 2,
wherein the mental fatigue calculation means is configured to calculate the mental fatigue degree by inputting the overall fatigue degree and the physical fatigue degree to a calculation engine,
the calculation engine being obtained by learning a model configured to calculate, when the overall fatigue degree and the physical fatigue degree are inputted thereto, the mental fatigue degree.

### [Supplementary Note 4]

The information processing device according to any one of Supplementary Notes 1 to 3, further comprising
an output control means configured to perform control to output information on the mental fatigue degree.

### [Supplementary Note 5]

The information processing device according to Supplementary Note 4,
wherein the output control means is configured to perform control to output an index based on the mental fatigue degree together with an index based on the overall fatigue degree and an index based on the physical fatigue degree. It is noted that examples of the “index based on the mental fatigue degree” include the mental fatigue degree and any other index (e.g., mental health level) calculated from the mental fatigue degree. It is also true for the “index based on the overall fatigue degree” and “index based on the physical fatigue degree”.

### [Supplementary Note 6]

The information processing device according to Supplementary Note 4 or 5,
wherein the output control means is configured to perform control to output advice to the object person determined based on the mental fatigue degree.

### [Supplementary Note 7]

The information processing device according to Supplementary Note 4,
wherein the output control means is configured to cause a display device to display information on a comparison between an index based on the mental fatigue degree and an index based on the mental fatigue degree of other members in a group to which the object person belongs. Examples of the “information on the comparison” include information indicative of the ranking, in the group, of the index based on the mental fatigue degree and a scatter diagram for comparing the index based on the mental fatigue degree in the group.

### [Supplementary Note 8]

The information processing device according to any one of Supplementary Notes 1 to 7,
wherein the mental fatigue calculation means is configured to calculate the mental fatigue degree based on an attribute of the object person, the overall fatigue degree, and the physical fatigue degree.

### [Supplementary Note 9]

A control method executed by a computer, the control method comprising:
acquiring an overall fatigue degree which indicates a degree of overall fatigue of an object person;
acquiring a physical fatigue degree which indicates a degree of physical fatigue of the object person; and
calculating a mental fatigue degree which indicates a degree of mental fatigue of the object person based on the overall fatigue degree and the physical fatigue degree.

### [Supplementary Note 10]

A storage medium storing a program executed by a computer, the program causing the computer to function as:
an overall fatigue acquisition means configured to acquire an overall fatigue degree which indicates a degree of overall fatigue of an object person;
a physical fatigue acquisition means configured to acquire a physical fatigue degree which indicates a degree of physical fatigue of the object person; and
a mental fatigue calculation means configured to calculate a mental fatigue degree which indicates a degree of mental fatigue of the object person based on the overall fatigue degree and the physical fatigue degree.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All patent and Non-Patent Literatures mentioned in this specification are incorporated by reference in its entirety.

### DESCRIPTION OF REFERENCE NUMERALS

1, 1A, 1B Information processing device
2 Input device
3 Display device
4 Storage device
6 Learning device
8 Terminal device
100, 100A Fatigue estimation system

What is claimed is:

1. A method using a device for calculating a degree of fatigue in communication with a sensor and a client device, comprising:
monitoring biological signal of an object person, by the sensor;
calculating, based on analysis results of the object person's saliva, an overall fatigue degree quantifying at least one of HHV-6 or HHV-7 of the object person, the analysis results being generated based on the biological signal;
acquiring a physical fatigue degree which indicates a degree of physical fatigue of the object person;
calculating a mental fatigue degree which indicate a degree of mental fatigue of the object person based on the overall fatigue degree and the physical fatigue degree;
generating display signal based on the mental fatigue degree of the object person; and
transmitting the display signal to the client device.

2. The method according to claim 1 comprising:
acquiring data indicating the response results of questionnaires by the object person; and
calculating, based on the data, the overall fatigue degree quantifying Chalder Fatigue Scale (CFS) of the object person.

3. The method according to claim 1 comprising,
acquiring fatigue related data related to fatigue of the object person; and
calculating the physical fatigue degree based on at least a part of the fatigue related data.

4. The method according to claim 1 comprising,
calculating the mental fatigue degree by inputting the overall fatigue degree and the physical fatigue degree to a calculation unit, the calculation unit being obtained by learning a model configured to calculate via machine learning, when the overall fatigue degree and the physical fatigue degree are inputted thereto, the mental fatigue degree.

5. The method according to claim 1 comprising,
generating the display signal based on information related to the mental fatigue degree.

6. The method according to claim 1 comprising,
generating the display signal based on the overall fatigue degree and the physical fatigue degree.

7. The method according to claim 1 comprising,
generating the display signal based on advice to the object person determined based on the mental fatigue degree.

8. The method according to claim 7,
wherein the advice includes one or more treatments optimizing at least one of the mental fatigue degree, the physical fatigue degree, and the overall fatigue degree.

9. The method according to claim 1 comprising,
generating the display signal based on display information on a comparison between an index based on the mental fatigue degree and an index based on the mental fatigue degree of other members in a group to which the object person belongs.

10. The method according to claim 1 comprising,
calculating the mental fatigue degree based on an attribute of the object person, the overall fatigue degree, and the physical fatigue degree.

11. A device for calculating a degree of fatigue in communication with a sensor and a client device, the device comprising:
at least one memory configured to store instructions; and
at least one processor configured to execute the instructions to:
calculate, based on analysis results of object person's saliva, an overall fatigue degree quantifying at least one of HHV-6 and HHV-7 of the object person, the analysis results being generated based on the biological signal monitored by the sensor;
acquire a physical fatigue degree which indicates a degree of physical fatigue of the object person;
calculate a mental fatigue degree which indicate a degree of mental fatigue of the object person based on the overall fatigue degree and the physical fatigue degree;
generate display signal based on the mental fatigue degree of the object person; and
transmit the display signal to the client device.

12. A non-transitory computer readable storage medium storing a program executed by a computer in communication with a sensor and a client device, the program causing the computer to:
calculate, based on analysis results of object person's saliva, an overall fatigue degree quantifying at least one of HHV-6 and HHV-7 of the object person, the analysis results being generated based on the biological signal monitored by a sensor,
acquire a physical fatigue degree which indicates a degree of physical fatigue of the object person;
calculate a mental fatigue degree which indicate a degree of mental fatigue of the object person based on the overall fatigue degree and the physical fatigue degree;
generate display signal based on the mental fatigue degree of the object person; and
transmit the display signal to a client device.
